(19) United States
(12) Patent Application Publication
(10) Pub. No.: US 2013/0172562 A1
(43) Pub. Date: Jul. 4, 2013

(54) PROCESS FOR THE PREPARATION OF A SINGLE ENANTIOMER OF 3-AMINOPIPERIDINE DIHYDROCHLORIDE

(71) Applicants: Graham Andrew Meek, Ely (GB); Syam Kumar Unniaran Purakkal Kunhimon, Thrissur (IN); R Shankar, Salem (IN); Vilas Hareshwar Dahanukar, Hyderabad (IN); TH. Krishna Mohan, Hyderabad (IN); Manoj Balu Wagh, Nashik (IN); Abir Kumar Pal, Birbhum (IN); V. Madhu Babu Meesala, East Godavari District (IN); Sonmit Shrivastava, Hyderabad (IN)

(72) Inventors: Graham Andrew Meek, Ely (GB); Syam Kumar Unniaran Purakkal Kunhimon, Thrissur (IN); R Shankar, Salem (IN); Vilas Hareshwar Dahanukar, Hyderabad (IN); TH. Krishna Mohan, Hyderabad (IN); Manoj Balu Wagh, Nashik (IN); Abir Kumar Pal, Birbhum (IN); V. Madhu Babu Meesala, East Godavari District (IN); Sonmit Shrivastava, Hyderabad (IN)

(73) Assignees: DR. REDDY'S LABORATORIES, INC., Bridgewater, NJ (US); DR. REDDY'S LABORATORIES LTD., Hyderabad (IN)

(21) Appl. No.: 13/704,142

(22) PCT Filed: Jun. 17, 2011
(86) PCT No.: PCT/US2011/040912

§ 371 Date: Mar. 5, 2013

Related U.S. Application Data
(60) Provisional application No. 61/355,692, filed on Jun. 17, 2010.

(51) Int. Cl. C07D 211/02

(52) U.S. Cl. CPC C07D 211/02
USPC 546/185

(57) ABSTRACT

A process comprising: (a) reduction of N-acetyl-3-aminopyridine (2): or its salt in the presence of hydrogen and a palladium catalyst deposited on solid support; (b) converting racemic N-acetyl-3-aminopiperidine (3) or its salt produced in step (a) to rac-3-aminopiperidine (rac-4) or its salt; (c) resolution of the racemic 3-aminopiperidine (rac-4) or its salt produced in step (b) with a chiral acid.

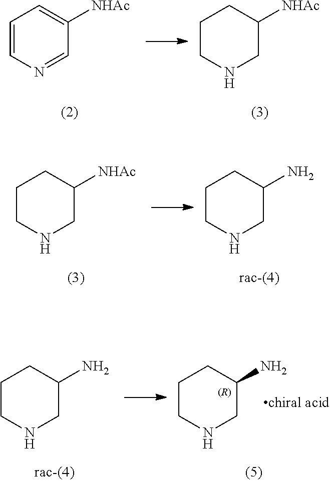

The application relates to processes for preparing either enantiomer of 3-aminopiperidine dihydrochloride with high % e.e. and processes for preparing either of the compounds (R)-piperidin-3-amine or (S)-piperidin-3-amine, with >98% e.e. The application specifically relates to a process for preparing (R)-3-aminopiperidine dihydrochloride with >98% e.e.

The salt 3-aminopiperidine dihydrochloride is an ingredient for several pharmaceutical agents. International Application No. WO/2007/075630 A1, published Jul. 5, 2007, and incorporated by reference in its entirety, describes the hydrogenation of 3-aminopyridine with supported rhodium catalyst, resolution with dibenzoyl tartaric acid, and acid exchange using hydrogen chloride in MTBE (methyl tert-butyl ether). This did not result in upgrading the enantiopurity and therefore the dibenzoyl tartaric acid salt of 3-aminopiperidine had to be repeatedly recrystallized prior to acid exchange. There is a need to identify a solvent system that would provide an enantiopurity upgrade at this stage thereby reducing the number of recrystallization steps in the process and improve the yield.

US 2006/0142310 describes the hydrogenation of 3-aminopyridine with 5 wt % of a mixed platinum/rhodium catalyst in acetic acid at 50° C. and 100 bar hydrogen pressure. Heterocycles, 1993, 36(10), 2383 describes use of samarium iodide in THF to reduce 3-aminopyridine to provide 3 products, of which 3-aminopiperidine is produced in 26% yield. Berichte der Deutschen Chemischen Gesellschaft [Abteilung] B: Abhandlungen, 1937, 70B, 635 describes the hydrogenation of 3-aminopyridine in methanol and hydrochloric acid catalyzed by platinum oxide WO 95/08536 describes the hydrogenation of several α,γ-dicarbonyl substituted 3-aminopyridine derivatives using platinum oxide in either acetic acid or methanol and hydrochloric acid. J. Med. Chem., 1980, 23, 848 describes the hydrogenation of 3-N-acetyl-aminopyridine using platinum oxide in methanol and concentrated hydrochloric acid in 47% yield following basification and isolation of 3-N-acetamido-piperidine. The reaction took 42 hours. Adv. Synth. Catal., 2008, 350, 807 describes kinetic resolution of 1-Boc-3-aminopiperidine with a transaminase enzyme in 42% yield with 97% e.e. The reaction was carried-out at a concentration of 10 mM with respect to 1-Boc-3-aminopiperidine to provide the (R) enantiomer. WO 2007/112368 describes a synthesis of (R)-3-aminopiperidine dihydrochloride from D-ornithine via esterification, cyclization to an amino amide, and reduction using lithium aluminium hydride at 60° C. followed by treatment with hydrochloric acid. This process involved a complicated precipitation of the hydrochloride salt. Synthetic Communications, 1998, 28, 3919 describes the synthesis of (R)-3-aminopiperidine dihydrochloride from D-glutamic acid. This involves esterification, amine protection, ester reduction with sodium borohydride/calcium chloride, activation with mesyl chloride, and subsequent displacement with benzylamine followed by benzyl group removal using palladium/carbon and hydrogen.

There remains a need to provide improved processes for preparing either single enantiomer of 3-aminopiperidine dihydrochloride, which upgrade the enantiomeric purity, eliminating the need for repeated recrystallization of the 3-aminopiperidine dibenzoyl tartaric acid salt, and which use a cheaper palladium catalyst.

### SUMMARY

In one aspect, the present application provides processes for the preparation of either enantiomer of 3-aminopiperidine dihydrochloride ((R)-4 or (S)-4), comprising acid exchange directly from the partially resolved 3-aminopiperidine chiral acid salt with hydrogen chloride in isopropyl alcohol/water as the solvent which occurs with enhancement of the chiral purity.

In another aspect the present application specifically relates to a process for preparing (R)-3-aminopiperidine dihydrochloride with >98% e.e. comprising acid exchange directly from the partially resolved 3-aminopiperidine chiral acid salt with hydrogen chloride in isopropyl alcohol/water as the solvent which occurs with enhancement of the chiral purity.

### DETAILED DESCRIPTION

In one aspect, the present application provides processes for the preparation of either enantiomer of 3-aminopiperidine dihydrochloride ((R)-4 or (S)-4), comprising acid exchange directly from the partially resolved 3-aminopiperidine chiral acid salt with hydrogen chloride in isopropyl alcohol/water as the solvent which occurs with enhancement of the chiral purity.

In another aspect, the present application provides processes for the preparation of (R)-3-aminopiperidine dihydrochloride ((R)-4) comprising acid exchange directly from the partially resolved 3-aminopiperidine dibenzoyl-(D)-tartaric acid salt (5) with hydrogen chloride in isopropyl alcohol/water as the solvent which occurs with enhancement of the chiral purity.

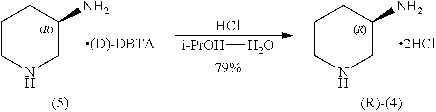

In one aspect, the present application further comprises neutralization of rac-3-aminopiperidine dihydrochloride (rac-4), without isolation, and formation of the 3-aminopiperidine dibenzoyl-(D)-tartaric acid salt (5). This reaction provides the diastereomeric salt with enhanced diastereomeric purity.

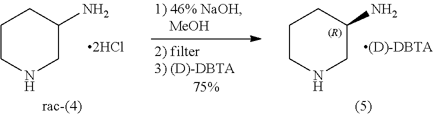

In one aspect, optionally the present application further comprises upgrade of the diastereoisomeric purity of 3-aminopiperidine dibenzoyl-(D)-tartaric acid salt (5).

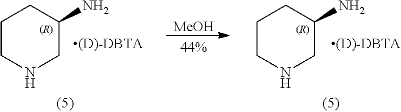

In one aspect, the present application further comprises hydrogenation of N-acetyl-3-aminopyridine (2), which is formed without isolation, to provide rac-N-acetyl-3-aminopiperidine acetate salt (3). The hydrogenation may be performed using a palladium catalyst on a solid support. The solid support maybe carbon, calcium carbonate, titania, or zirconia. In one embodiment, the hydrogenation may be performed in the presence of palladium on carbon.

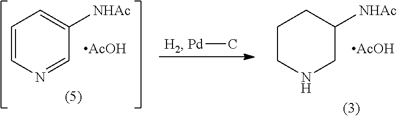

In one aspect, the present application further comprises formation of N-acetyl-3-aminopyridine (2) in situ from 3-aminopyridine (1).

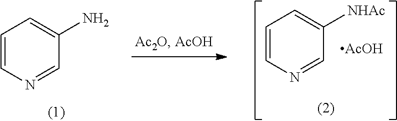 [SMILES: "·AcOH" ; Nc1cccnc1 ; CC(=O)Nc1cccnc1]

In one aspect, the present application further comprises formation of rac-3-aminopiperidine dihydrochloride rac-(4) by acidic hydrolysis of the acetyl group in rac-N-acetyl-3-aminopiperidine acetate salt (3) and subsequent azeotropic drying with ethanol.

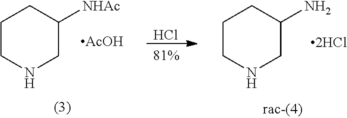 [SMILES: "·AcOH" ; CC(=O)NC1CCCNC1 ; Cl ; NC1CCCNC1 ; "·2HCl"]

The acid exchange reactions are usually done with from about 2 to about 10 molar equivalents of hydrochloric acid, typically in the range of about 2 to about 4 molar equivalents of hydrochloric acid. In one embodiment the molar equivalents of hydrochloric acid are at least about 2, and in another embodiment at least about 3. However the reaction can also be performed with molar equivalents of hydrochloric acid as high as about 5.

The upgrade of the diastereoisomeric purity of 3-aminopiperidine dibenzoyl-(D)-tartaric acid salt (5) is optionally done with an amount of an alcohol solvent with respect to (5) from about 5 v/w to about 50 v/w, typically with about 10 v/w to about 25 v/w. In one embodiment the amount of alcohol solvent with respect to (5) is at least 20 v/w, and in another embodiment at least about 25 v/w. However the reaction can also be performed with an amount of alcohol solvent with respect to (5) as high as about 30. In one embodiment the alcohol solvent is methanol. The reaction time to upgrade the diastereoisomeric purity of 3-aminopiperidine dibenzoyl-(D)-tartaric acid salt (5) is typically from about 0.5 hours to about 48 hours. In one embodiment the time to upgrade the diastereoisomeric purity is from about 1 hour to about 24 hours, and at least about 2 hours.

Suitable bases for the neutralization step include sodium hydroxide, potassium hydroxide, sodium carbonate, potassium carbonate lithium hydroxide, and the like. The neutralization reactions usually employ from about 1.0 to about 4.0 molar equivalents of suitable base with respect to rac-4. In one embodiment the molar equivalents of suitable base with respect to rac-4 are about 2.0 to about 3.0, and in another embodiment at least about 2.05 molar equivalents of suitable base with respect to rac-4. The neutralization reactions usually employ from about 5 to about 30 v/w of an amount of methanol with respect to rac-4. In one embodiment from about 10 to about 20 v/w of an amount of methanol with respect to rac-4, and in another embodiment about 14.5 v/w of an amount of methanol with respect to rac-4. Suitable acids for the resolution reactions include (D)-DBTA or any of the chiral, non-racemic acids described in WO 2007/078630. The resolution reactions employ typically from about 0.5 to about 4.0 molar equivalents of suitable chiral, non-racemic acids such as (D)-DBTA with respect to rac-4. In one embodiment about 0.5 to about 2.0 molar equivalents of suitable chiral, non-racemic acids such as (D)-DBTA with respect to rac-4, with at least 1.07 molar equivalents of suitable chiral, non-racemic acids such as (D)-DBTA with respect to rac-4. The resolution reactions are usually heated above room temperature, typically in the range of about 44° C. to about 84° C. In one embodiment the reaction temperature is about 54° C. to about 74° C. In one embodiment the temperature is raised to at least about 60° C., and in another embodiment to at least about 64° C. However the reaction can also be performed at temperatures as high as about 80° C. The time of the resolution reaction is typically from about 0.5 hours to about 48 hours. In one embodiment the time is about 1 hour to about 24 hours, and at least about 2 hours.

The hydrogenation reactions are usually done above atmospheric pressure, typically in the range of about 2 bar to about 500 bar. In one embodiment the pressure is about 5 bar to about 100 bar. In one embodiment the pressure is raised to at least about 20 bar, and in another embodiment the pressure is raised to at least about 10 bar. However the reaction can also be performed at pressures as high as about 90 bar. The hydrogenation reactions are usually done with a Pd/C loading with respect to (2), typically in the range from about 0.5 wt % to about 200 wt %. In one embodiment the Pd/C loading is about 1 wt % to about 100 wt %. In one embodiment the Pd/C loading with respect to (2) is least about 5 wt %, and in another embodiment to at least about 3.5 wt %. However the reaction can also be performed at a Pd/C loading with respect to (2) as high as about 50 wt %. The hydrogenation reactions are usually heated above room temperature, typically in the range of about 20° C. to about 140° C. In one embodiment the temperature is about 25° C. to about 120° C. In one embodiment the temperature is raised to at least about 60° C., and in another embodiment to at least about 80° C. However the reaction can also be performed at temperatures as high as about 100° C. The time of the hydrogenation reactions is typically from about 3 hours to 7 days. In one embodiment the time is from about 1 hour to about 24 hours, and in another embodiment at least about 3 hours. Suitable solvents for the hydrogenation reactions include acetic acid, propionic acid, butanoic acid, or any carboxylic acid that is a liquid under the reaction conditions.

Suitable reagents for the in situ acylation reactions include acetic anhydride, acetyl chloride, or any carboxylic acid chloride, propionic anhydride, butanoic anhydride, or any carboxylic acid anhydride. Suitable solvents for the in situ acylation reactions include acetic acid, propionic acid, butanoic acid, or any carboxylic acid that is a liquid under the reaction conditions. The in situ acylation reactions are usually cooled below room temperature, typically in the range of about 0° C. to about 25° C. In one embodiment the temperature is about 10° C. to about 25° C. However the reaction can also be performed at temperatures as high as about 120° C. The in situ acylation reactions are usually done with from about 1.0 to about 20 molar equivalents of acylating agent, typically in the range of about 1 to about 10 molar equivalents of acylating agent. In one embodiment from about 1 to about 5 molar equivalents of acylating agent are used. In one embodiment the molar equivalents of acylating agent are at least about 1.0, and in another embodiment at least about 1.05. However the reaction can also be performed with molar equivalents of acylating agent as high as about 2.5. The in situ acylation reactions are usually done at about 2 v/w to about 10 v/w concentration of (1) in acetic acid. In one embodiment the concentration of (1) in acetic acid is about 4 v/w. The time of the in situ acylation reactions is typically from about 1 hour to 2 days. In one embodiment from about 2 hour to about 24 hours, and in another embodiment is about 2 hours.

Suitable acids for the acidic hydrolysis reactions include hydrochloric acid, sulfuric acid, hydrobromic acid, phosphoric acid, tetrafluoroboric acid, hydrofluoric acid, hydriodic acid, perchloric acid, or any suitable inorganic acid. Suitable solvents for the acidic hydrolysis reactions include ethanol, methanol, 2-propanol, or any suitable alcohol solvent. The acidic hydrolysis reactions are typically done with an acid strength in the range of about 0.5 M to about 12 M. In one embodiment the acid strength is about 1 M to about 12 M. In one embodiment the acid strength is at least about 3 M, and in another embodiment at least about 6 M. However, the reaction can also be performed at acid strengths as high as about 10 M. The acidic hydrolysis reactions are usually done with from about 1 to about 30 molar equivalents of acid, typically in the range of about 1 to about 20 molar equivalents of acid. In one embodiment the molar equivalents of acid are at least about 2, and in another embodiment at least about 3. However the reaction can also be performed with molar equivalents of acid as high as about 5. The volume of alcohol with respect to (3) used each time in the acidic hydrolysis reactions is typically from about 1 v/w to about 20 v/w. In one embodiment the volume of alcohol with respect to (3) is about 1.2 v/w. The number of alcohol dissolution/concentration cycles used in the acidic hydrolysis reactions is typically from about 1 to about 10. In one embodiment the number of alcohol dissolution/concentration cycles used is about 3.

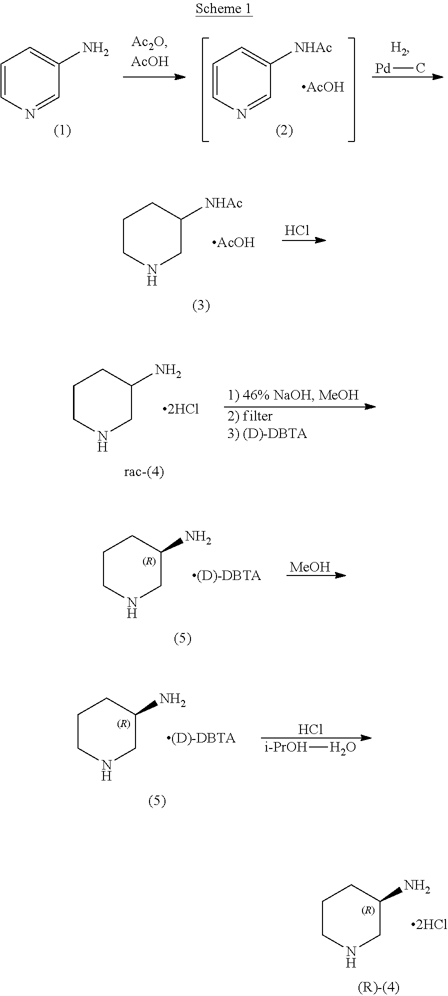

Scheme 1 outlines the synthesis of the Examples of the present application.

The present process has fewer recrystallization steps, involves the use of a less expensive catalyst such as Pd/C, lower pressure, produces a single product in higher yield, and is a quicker reaction. The present process provides either enantiomer of the product by switching resolving agent whereas transaminase route needs to find (S)-selective enzyme. The present route is carried-out at practical, industrially favored concentrations unlike the transaminase route, and allows for good material throughput.

### DEFINITIONS

The following definitions are used in connection with the present application unless the context indicates otherwise. Celite™ is flux-calcined diatomaceous earth. Celite™ is a registered trademark of World Minerals Inc. DBTA is dibenzoyl-tartaric acid, HPLC is high-pressure liquid chromatography, MeOH is methanol, and CROWNPAK™ CR refers to HPLC columns containing a chiral crown ether as a chiral selector which is coated onto 5 μm silica. Crownpak™ is a registered trademark of DAICEL CHEMICAL. INDUSTRIES, LTD. The term “% e.e.” means the enantiomeric excess of a substance, which is defined as the absolute difference between the mole fraction of each enantiomer. The term “de” means “diastereomeric excess”, the excess of one diastereomeric pair of enantiomers over the other pair of enantiomers (assuming two asymmetric centers) and NMR is nuclear magnetic resonance. As used herein, the term “reacting” is intended to represent bringing the chemical reactants together under conditions such to cause the chemical reaction indicated to take place.

An “alcohol solvent” is an organic solvent containing a carbon bound to a hydroxyl group. “Alcohol solvents” include but are not limited to methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, C₁₋₆alcohols, or the like.

A “chiral acid” is commonly used for the resolution of nitrogen containing compounds. “Chiral acids” include but are not limited to (1R or 1S)-10-camphorsulfonic acid, (D or L)-tartaric acid, (D or L)-dibenzoyl tartaric acid, (1R or 1S)-3-bromocamphor-10-sulfonic acid, (R or S)-1,1″-binaphthyl-2,2″-diyl-hydrogenphosphate, (D or L)-di-O,O′-p-toluoyl-tartaric acid, (D or L)-di-O,O′-o-toluoyl-tartaric acid, (D or L)-N-acetyl-phenylalanine, (D or L)-acetylmandelic acid, (R or S)-cyclohexylphenylglycolic acid, (S)-camphanic acid, (R or S)-2-pyrrolidone-5-carboxylic acid, naproxen, ibuprofen; (D or L)-tartaric acid, (D or L)-malic acid, L-lactic acid, (R or S)-3-hydroxybutyric acid, or hyodeoxycholic acid.

Comparison of hydrogenation of 3-aminopyridine in acetic acid and 3-N-acetylaminopyridine in acetic acid illustrating the higher reactivity of the N-acetyl derivative.

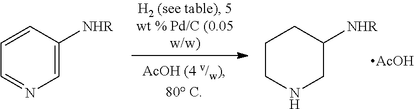

| | | H₂ Pressure | | |
|---|---|---|---|---|
| Entry | R | (bar) | Time (hrs) | Conv. (%)^{a} |
| 1 | H | 20 | 18 | 50 |
| 2 | Ac | 10 | 5 | >98 |

^{a}Determined by ¹H NMR

Procedures used to perform the processes of the present application are illustrated in the examples. Reasonable variations of the described procedures are intended to be within the scope of the present invention.

### EXAMPLES

### Example 1

### Preparation of rac-3-aminopiperidine dihydrochloride (rac-4)

Acetic anhydride (91.63 g, 897.5 mmol) was added by drops over 10 minutes to a solution of 3-aminopyridine (1, 80.48 g, 855.2 mmol) in acetic acid (400 ml) cooled to 10° C. An exotherm of 15° C. was observed. Once the addition was complete, the solution was stirred at room temperature for 2 hours and then added to a glass liner along with 5% palladium/carbon (12.24 g). After securing in a pressure vessel, the solution was charged with nitrogen to a pressure of 10 bar, stirred until equilibrated and then vented. This nitrogen charge/stir/vent cycle was repeated two times. The vessel was then charged with hydrogen to a pressure of 10 bar and vented, without stirring. This hydrogen charge/vent cycle was repeated two times. The vessel was then charged to 10 bar of hydrogen pressure, heated to 80° C. and stirred, with the pressure being maintained between 9.9 and 10 1 bar. After 3 hours hydrogen consumption had ceased. The contents were cooled to room temperature and the vessel was charged with nitrogen to a pressure of 10 bar, stirred for 20 minutes, and then vented. This nitrogen charge/stir/vent cycle was repeated one more time and the contents were then filtered through Celite™ and washed with acetic acid (40 ml). The filtrate was partially concentrated in vacuo to a bulk weight of 284.04 g (61 wt % solution in acetic acid assuming 100% conversion/yield). Hydrochloric acid (428 ml of a 6M solution, 2568 mmol) was added to the solution in acetic acid and heated at reflux for 18 hours. The solution was cooled to room temperature and concentrated in vacuo. Ethanol (200 ml) was charged to the residue which was subsequently concentrated in vacuo. This ethanol charge/concentration process was repeated a further two times with the same quantity of solvent to provide the title compound as a white solid (120.6 g, 81%). ¹H NMR (DMSO-d₆, 400 MHz) δ ppm 9.50-8.50 (4H, br), 3.48-3.42 (2H, m), 3.39-3.30 (1H, br), 3.19 (1H, br d, J 12 Hz), 2.90 (1H, t, J=12 Hz), 2.77 (1H, td, J 12 and 3 Hz), 2.05 (1H, br d, J=10 Hz), 1.88 (1H, dt, J 12 and 3 Hz), 1.78-1.69 (1H, m) and 1.61 (1H, qd, J 12 and 3 Hz).

### Example 2

### Preparation of (R)-3-aminopiperidine dibenzoyl-(D)-tartaric acid salt (5)

Sodium hydroxide (10.3 g of a 46-48% solution, 4.74 g a.i., 118.5 mmol) was added by drops to an ice-water bath cooled suspension of rac-3-aminopiperidine dihydrochloride (rac-4, 10.0 g, 57.8 mmol) in methanol (145 ml). Once the addition was complete the solution was stirred at room temperature for one hour and then filtered (through porosity #3 filter paper: 4.90 g sodium chloride collected, 71% of theory) and the solid was washed with methanol (2 ml). Dibenzoyl-(D)-tartaric acid (22.16 g, 61.84 mmol) was then added to the solution which was subsequently heated to 60° C. (very gentle reflux) for 2 hours. The resultant suspension was cooled to 20° C. over 1-2 hours and then stirred at this temperature for 20 hours. The solid was collected by filtration and sequentially washed with a mixture of methanol/water (19 ml/1 ml), then methanol (20 ml) and dried in vacuo to provide the title compound as a white solid (19.9 g, 75%) with 13.2% de.

### Example 3

### De upgrade of (R)-3-aminopiperidine dibenzoyl-(D)-tartaric acid salt (5)

A suspension of (R)-3-aminopiperidine dibenzoyl-(D)-tartaric acid salt (5, 18.6 g, 40.6 mmol, 13.2% de) in methanol (465 ml) was heated to 60° C. for 2 hours (very gentle reflux) and then cooled to 20° C. over 1-2 hours before stirring at this temperature for 19 hours. The suspension was filtered and the residue was washed with fresh methanol (2×18 ml) before being dried in vacuo to provide the title compound as a white solid (8.28 g, 44%) with 96.5% de.

### Example 4

### Preparation of (R)-3-aminopiperidine dihydrochloride (R-4)

Hydrogen chloride (7.2 ml of a 5-6M solution in 2-propanol, 36 mmol assuming 5M) was added by drops to a suspension of (R)-3-aminopiperidine dibenzoyl-(D)-tartaric acid salt in 2-propanol (5, 30 ml) and water (1.9 ml) at 30° C. After stirring at this temperature for one hour, the mixture was heated to 60° C. to provide a clear solution. After stirring at this temperature for 90 minutes, the solution was cooled to 20° C. over 1-2 hours and then stirred at this temperature for 18 hours. The solid was collected by filtration under vacuum with a flow of nitrogen and then sequentially washed with a mixture of 2-propanol/water (2.1 ml/0.1 ml) followed by 2-propanol (3×2.2 ml). The filtration and washing operations were carried-out under vacuum and a flow of nitrogen. The solid was dried in vacuo (50° C., 12 mbar) to provide the title compound as a white solid (1.64 g, 79%) with 99.6% e.e. ¹H NMR (DMSO-d₆, 400 MHz) δ ppm 7.97 (4H, d, J 8 Hz), 7.64 (2H, t, J=8 Hz), 7.52 (4H, t, J=8 Hz), 5.61 (2H, s), 3.06 (2H, br d, J=9 Hz), 2.79 (1H, br d, J=12 Hz), 2.63 (1H, dd, J 12 and 10 Hz), 2.48-2.43 (1H, m), 1.76-1.67 (1H, m), 1.54-1.42 (1H, m) and 1.37-1.23 (2H, m).

### Example 5

### Preparation of rac-3-aminopiperidine dihydrochloride (rac-4)

Acetic anhydride (65.1 g, 638 mmol) was added dropwise over 10 minutes to a solution of 3-aminopyridine (50.0 g, 531 mmol) in acetic acid (150 ml) cooled to 10° C. An exotherm of 15° C. was observed. Once the addition was complete the solution was stirred at room temperature for 2 hours and then added to a glass liner along with 10% palladium/carbon (2.5 g). After securing in a pressure vessel, the solution was charged with nitrogen to a pressure of 10 bar, stirred until equilibrated, and then vented. This nitrogen charge/stir/vent cycle was repeated two times. The vessel was then charged with hydrogen to a pressure of 1-2 bar and vented, without stirring. This hydrogen charge/vent cycle was repeated two times. The vessel was then charged to 10 bar, heated to 80° C. and stirred, with the pressure being maintained between 16-20 bar. After 10 hours hydrogen consumption ceased. The contents were cooled to room temperature and the vessel was charged with nitrogen to a pressure of 10 bar, stirred for 20 minutes, and then vented. This nitrogen charge/stir/vent cycle was repeated one more time and the contents were then filtered through Celite™ and washed with acetic acid (12.5 ml). The filtrate was completely concentrated in vacuo to a bulk weight of 150 g. Hydrochloric acid (125 ml, 35% solution) was added to the solution in acetic acid and heated at reflux for 12 hours. The solution was cooled to room temperature and concentrated in vacuo. Isopropyl alcohol (150 ml) was charged to the residue which was subsequently concentrated in vacuo. This Isopropyl alcohol charge/concentration process was repeated a further two times with the same quantity of solvent to provide the title compound as a white solid (80.5 g, 87%).

### Example 6

### Preparation of (R)-3-aminopiperidine dibenzoyl-(D)-tartaric acid salt (5)

Sodium hydroxide (44.3 g of a 10-12% solution, 4.74 g al, 118.5 mmol) was added in drops to an ice-water bath cooled suspension of rac-3-aminopiperidine dihydrochloride (10.0 g, 57.8 mmol) in methanol (70 ml). Once the addition was complete the solution was stirred at room temperature for one hour, filtered through porosity #3 filter paper (4.90 g sodium chloride collected, 71% Th.), and the solid was washed with methanol (10 ml). Dibenzoyl-(D)-tartaric acid (22.16 g, 61.84 mmol) was then added to the solution which was subsequently heated to 60° C. (very gentle reflux) for 2 hours. The resultant suspension was cooled to 20-25° C. over 1-2 hours and then stirred at 20-25° C. for 8 hours. The reaction mixture was then cooled further to −10 to −5° C. and stirred at this temperature for 8 hours. The solid was collected by filtration and washed with a methanol (10 mL) and dried in vacuo to provide the title compound as a white solid (10.8 g, 41%) with 93% de.

### Example 7

### Preparation of (R)-3-aminopiperidine dihydrochloride (R-4)

Hydrogen chloride (7.2 ml of a 5-6M solution in isopropyl alcohol, 36 mmol assuming 5M) was added in drops to a suspension of (R)-3-aminopiperidine dibenzoyl-(D)-tartaric acid salt (5.5 g, 12 mmol) in isopropyl alcohol (30 ml) and water (1.65 ml) at 30° C. After stirring at this temperature for one hour, the mixture was heated to 60° C. After stirring at this temperature for 90 minutes, the solution was cooled to 20° C. over 1-2 hours and then stirred at this temperature for 18 hours. The solid was collected by filtration under vacuum with a flow of nitrogen and then sequentially washed with a mixture of isopropyl alcohol (5.5 ml). The filtration and washing operations were carried-out under vacuum and a flow of nitrogen. The solid was dried in vacuo (50° C., 12 mbar) to provide the title compound as a white solid (1.7 g, 82%) with 99.0% e.e.

Enantiopurity assay for (R)-3-Aminopiperidine dihydrochloride (R-4) and for inferred de determination of (R)-3-aminopiperidine dibenzoyl-(D)-tartaric acid salt (5).

HPLC Conditions
Column: Crownpak™ CR+ (150×4.6 mm)
Mobile phase: 95:5 v/v pH 1 HClO₄:MeOH (16.27 g 70% HClO₄->1 L H₂O=pH1)
Flow rate: 0.6 ml/min
Column Temp. 0° C.
Detection: Refractive index (Gilson 133 sensitivity 2)
Injection: 5 uL
Sample prep. ˜5 mg to 300 uL MeOH then add 700 uL of pH1 HClO₄

Retention Times

| | |
|---|---|
| (S) | 3.0 minutes |
| (R) | 3.7 minutes |

The compounds herein described have asymmetric centers. Compounds of the present application containing an asymmetrically substituted atom may be isolated in optically active or racemic forms. It is well known in the art how to prepare optically active forms, such as by resolution of racemic forms or by synthesis from optically active starting materials. The structure depicted for the compounds within the present application are also meant to include all isomeric (e.g., enantiomeric) forms of the structures. For example, both the R and the S configurations at the stereogenic carbon are included in this application.

The structure depicted for the compounds within the present application are also meant to include all isomeric (e.g., enantiomeric or conformational) forms of the structures. For example, both the R and the S configurations at the stereogenic carbon are included in this application. Therefore, single stereochemical isomers as well as enantiomeric and conformational mixtures of the present compound are within the scope of the application. It is well known in the art how to prepare optically active forms, such as by resolution of racemic forms or by synthesis from optically active starting materials. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this application.

Throughout this application, various publications are referenced. The disclosures of these publications in their entireties are hereby incorporated by reference into this application in order to more fully describe the state of the art as known to those skilled therein as of the date of the application described and claimed herein.

While particular embodiments of the present application have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A process comprising:
(a) reduction of N-acetyl-3-aminopyridine (2): 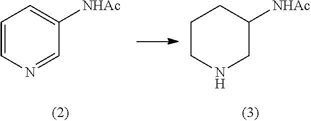 [SMILES: CC(=O)Nc1cccnc1 ; CC(=O)NC1CCCNC1]
or its salt in the presence of hydrogen and a palladium catalyst deposited on solid support;
(b) converting racemic N-acetyl-3-aminopiperidine (3) or its salt produced in step (a) to rac-3-aminopiperidine (rac-4) or its salt; 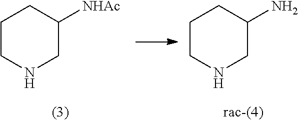 [SMILES: CC(=O)NC1CCCNC1 ; NC1CCCNC1]
(c) resolution of the racemic 3-aminopiperidine (rac-4) or its salt produced in step (b) with a chiral acid

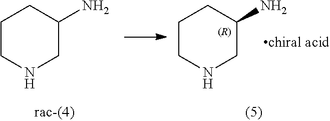

2. The process of claim 1, wherein the solid support for palladium is carbon, calcium carbonate, titania, or zirconia.

3. The process of claim 3, wherein the solid support is carbon.

4. The process of claim 1, wherein the hydrogen pressure is above atmospheric.

5. The process of claim 4, wherein the hydrogen pressure is from about 2 bar to about 500 bar.

6. The process of claim 5, wherein the hydrogen pressure is at least about 10 bar.

7. The process of claim 1, wherein the chiral acid is in step (c) is dibenzoyl-(D)-tartaric acid and the salt formed is the 3-aminopiperidine dibenzoyl-(D)-tartaric acid salt (5):

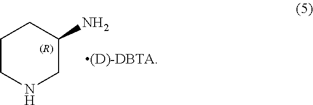

8. The process of claim 1, further comprising formation of N-acetyl-3-aminopyridine (2): 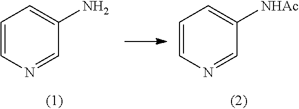 [SMILES: Nc1cccnc1 ; CC(=O)Nc1cccnc1]
or a salt thereof from 3-aminopyridine (1) by reaction with an acetylating agent.

9. The process of claim 8, wherein the acetylating agent is acetic anhydride, acetyl chloride, or a mixture thereof.

10. The process of claim 9, wherein the acetylating agent is acetic anhydride.

11. The process of claim 7, optionally further comprising upgrade of the diastereoisomeric purity of 3-aminopiperidine dibenzoyl-(D)-tartaric acid salt (5): 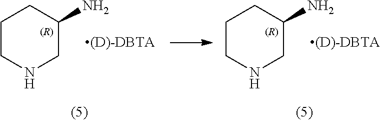
by heating in an alcohol solvent.

12. The process of claim 11, wherein the alcohol solvent is methanol.

13. The process of claim 7, further comprising acid exchange directly from the partially resolved 3-aminopiperidine dibenzoyl-(D)-tartaric acid salt (5): 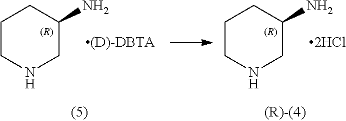
with hydrogen chloride in isopropyl alcohol/water as the solvent.

14. The process of claim 8, wherein the acetylating of 3-aminopyridine (1) is performed in the presence of acetic acid, propionic acid, or butanoic acid.

15. The process of claim 14, wherein the acetylating of 3-aminopyridine (1) is performed in the presence of acetic acid.

16. The process of claim 8, wherein the salt of N-acetyl-3-aminopyridine (2) is the acetate, propionate, or butanoate.

17. The process of claim 16, wherein the salt of N-acetyl-3-aminopyridine (2) is acetate salt.
